(19) United States
(12) Patent Application Publication
(10) Pub. No.: US 2008/0262625 A1
(43) Pub. Date: Oct. 23, 2008

(54) Medical Prosthetic Devices Presenting Enhanced Biocompatibility and Wear Resistance, Based On Colbalt Alloys and Process for Their Preparation

(76) Inventors: Silvia Spriano, Torino (IT); Sante Bugliosi, Torino (IT)

Correspondence Address: SUGHRUE MION, PLLC, 2100 PENNSYLVANIA AVENUE, N.W., SUITE 800, WASHINGTON, DC, 20037, US

(21) Appl. No.: 11/576,827

(22) PCT Filed: Oct. 6, 2005
(86) PCT No.: PCT/IB05/53291

§ 371 Date: Feb. 20, 2008

(30) Foreign Application Priority Data
Oct. 8, 2004 (IT) TO2004A000692

(51) Int. Cl. A61F 2/32

(52) U.S. Cl. 623/22.15

(57) ABSTRACT

Medical prosthetic devices and in particular femoral head and/or acetabular cup of articular prostheses, made out of a cobalt alloy are submitted to a surface modification in order to obtain the formation of a thin surface layer constituted by Co—Ta and or Co—Nb intermetallic compounds, with the aim of inducing to the prosthetic device enhanced characteristics of biocompatibility, low metal ion release, higher hardness and wear resistance. The process of surface modification is performed by a treatment of the alloy in a molten salt mixture containing a tantalum halide, without applying any electrical field.

The present invention concerns medical prosthetic devices, or medical implants, consisting of cobalt or cobalt alloy, presenting enhanced characteristics of biocompatibility, hardness and wear resistance, and it refers to a process for their preparation.

In the prosthetic field, the surfaces coupling in the movement of an artificial articular joint (hip and knee joints) are in direct contact, presenting significant friction and wear. Co—Cr—Mo—Ni alloys are widely used for articular prostheses, in particular in total hip joints, with a metal-on-metal-contact. They produce, inside the artificial joint, a moderate, but measurable amount of wear debris.

It must be considered that this debris presents potentially a high toxicity, in fact it can release Cr, Co and Ni as metal ions presenting different oxidation grade, being in contact for a long time with physiological fluids.

Some data reported in literature underline the potential danger ability of metallic wear debris, even if in reduced amount (G. Gasparini et al.^{[1]}). They arouse a typical reaction in the organism. A homogeneous population of cells is formed and they are activated by phagocitosis, because of the reduced dimensions (20-100 nm) of metallic wear debris. They can release factors which induce osteolitic phenomena through the activation of osteoclasts.

Furthermore frequent cases of cellular necrosis are revealed when wear debris production is especially high during a reduced time and they can enhance osteolitic phenomena.

Certainly the released ions interfere with the cellular metabolism and they cause toxic and mutagen biochemical reactions, in some cases also of the immune system, up to vascular damage with bony necrosis and the consequent prosthetic failure. The eventual correlation between the presence of metallic ions and the frequency of malignant tumours is still dubious (P. Rossi et al.^{[2]}).

The haematic and urinary concentrations of Co—Cr—Ni—Mo in thirty patients presenting a hip joint were analysed in a recent paper written by Massè et al.^{[3]}. The work underlines a relevant increment in the concentration of Co and Cr in patients with a metal-on-metal prosthesis.

Considering the composition, niobium and tantalum are promising elements in order to obtain a highly compatible surface (D. M. Findlay et al.^{[4]}). In fact they show an exceptional corrosion resistance and, in particular, tantalum was recently used with success for osteointegrated devices in the prosthetic field.

In fact it can be found on the marketing several devices made out of porous tantalum, containing 99 wt % of tantalum and 1 wt % of amorphous carbon, used as acetabular caps or as scaffolds for hip reconstruction when a relevant amount of bone was lost (X. Zou et al.^{[5]}).

Some investigations showed that tantalum do not induce cytotoxic phenomena and that osteoblastic cells adhere, proliferate and differentiate easily on it (D. M. Findlay et al.^{[6]}).

Other in-vivo investigations on animals showed that there is no trace of metal, as metallic ions, in animal tissues around the implant, confirming its high corrosion resistance and low metal ion release (H. Matsuno et al.^{[7]}).

EP-A-0 555 033 describes medical implants made out of a titanium, zirconium or cobalt alloy, containing up to 2 wt % of a easily oxidisable or nitridable metallic solute, as tantalum, and presenting a surface layer of the implant, where this solute is oxidised or nitridated through internal oxidation or nitring, with the aim of inducing an enhanced surface hardness and abrasion resistance.

The described process so requires the use of modified bulk metal alloys to include said solute.

The present invention supplies prosthetic devices or medical implants made out of conventional cobalt alloys on the marketing and showing enhanced biocompatible characteristics, low metal ion release, higher hardness and good tribological behaviour, because of a surface modification treatment that induces the formation of intermetallic compounds and the formation of a surface diffusion layer of tantalum and/or niobium on the alloy.

WO02/068007 and WO02/068729 describe surface modified biomedical implants with tantalum, by using electrodeposition processes from molten salts or by CVD deposition. However, in this case the surface shows an external layer of Alfa-tantalum presenting high ductility, that is absent in the present invention, that furthermore uses a process in molten Salts without applying any electrical field.

So it is an object of the present invention a medical prosthetic device or a medical implant comprising a bulk made out of cobalt or cobalt alloy, characterised by a thin surface layer, with an enrichment of tantalum and/or niobium in the composition, respect to the composition of the bulk material, and presenting intermetallic compounds which are rich in tantalum.

More characteristics of the devices are described in the included dependent claims.

The preferred process for the production of the prosthetic devices, that is a further object of the invention, includes a treatment of the cobalt or cobalt alloy in a mixture of molten salts, without applying any electrical field, comprising a tantalum and/or niobium halide, eventually added—respectively—with metal tantalum and/or niobium.

This process according to the invention must be preferred, because it has the advantage of inducing a surface modification presenting a strong interface between the surface and the bulk, through the presence of a diffusion layer.

The process of treatment in molten salts is preferably performed by using a mixture containing between 20 wt % and 100 wt % of a tantalum and/or niobium halide and/or by using between 0 wt % and 80 wt % of an alkaline or alkaline earth halide.

The tantalum halide is preferably K₂TaF₇, but also others salts containing tantalum can be used, such as fluorides (TaF₃ [TaF₅]₄), chlorides (TaCl₃, TaCl₄, TaCl₅), bromides (TaBr₃, TaBr₄, TaBr₅) and iodides (TaI₄, TaI₅). Analogous compounds can be used in the case of niobium.

The alkaline or alkaline earth halide is preferably sodium chloride, but other chlorides, bromides or iodides of Na, K, Li, Ca e Mg can be used.

Preferably, the bath of molten salts is added by pure tantalum and/or niobium, as metal powder, by using a concentration up to 20 wt % and more preferably lower than 5 wt %, respect to the composition of the mixture of molten salts.

The treatment is performed at a temperature generally comprised between 700° C. and 1500° C., preferably higher than 800° C. (in any case lower than the melting temperature of cobalt or of the cobalt alloy employed), for a time between 15 minutes and 8 hours.

The bulk material can be pure cobalt or a cobalt alloy. Typically the cobalt alloys employed for the production of prosthetic devices include cobalt in an amount higher than 40 wt %, for instance 45-70 wt %, typically alloyed with chromium (15-30 wt %) and molybdenum (4-10 wt %), with other eventual components as nickel, iron, silicon, manganese and carbon.

The nominal composition is reported in the table below according to ISO 5832, referred to the cobalt alloys for medical implants, usable as an example in the field of the invention.

| | | | Stellite 25 | |
|---|---|---|---|---|
| Name of the | Stellite 21 | | ASTM F90 | MP 35 N |
| alloy | ASTM F75 | ASTM F799 | L-605 | ASTM F562 |
| ISO code | 5832-4 | 5832-12 | 5832-5 | 5832-6 |
| Co | bal (58-69) | bal (58-69) | bal | bal |
| Cr | 26.5-30 | 26.5-30 | 19-21 | 20 |
| W | — | — | 14-16 | — |
| Mo | 4.5-7 | 4.5-7 | — | 10 |
| C | 0.25 | 0.25 | 0.10 | — |
| Fe | 1 (max) | 1 (max) | 3 (max) | — |
| Ni | 1 (max) | 1 (max) | 9-11 | 35 |
| Si | 2 (max) | 2 (max) | 1 (max) | — |
| Mn | 1 (max) | 1 (max) | 2 (max) | — |

bal = balance to 100%

It is intended that cobalt alloys containing low amounts of niobium and/or tantalum (for instance lower than 2 wt %) can be used in the field of the invention; however the invention does not require the presence of said metals in the implant bulk material.

According to the invention, the bulk of the cobalt or cobalt alloy material constituting the implant shows a thin surface layer that is strongly enriched of tantalum or niobium, as components of intermetallic compounds, respect to the bulk material.

The surface layer shows a hardness significantly higher and a wear resistance significantly higher than the (untreated) bulk material.

Said surface layer can be 0.5-40 μm thick, particularly lower than 10 μm. The tantalum or niobium concentration inside the layer changes versus the layer thickness, because it is a diffusion layer, and it can reach surface values of 90 wt % of tantalum and niobium, in any case it is higher than 5 wt %. The surface concentration of tantalum or niobium is typically about 70-90 wt %.

Said surface layer includes tantalum and/or niobium as components of Co—Ta intermetallic compounds, as for instance Co₂Ta, Co₃Ta, Co₅Ta, CoTa, CoTa₂, CoTa₃ and analogous intermetallic compounds.

In the attached figures, concerning the following example:

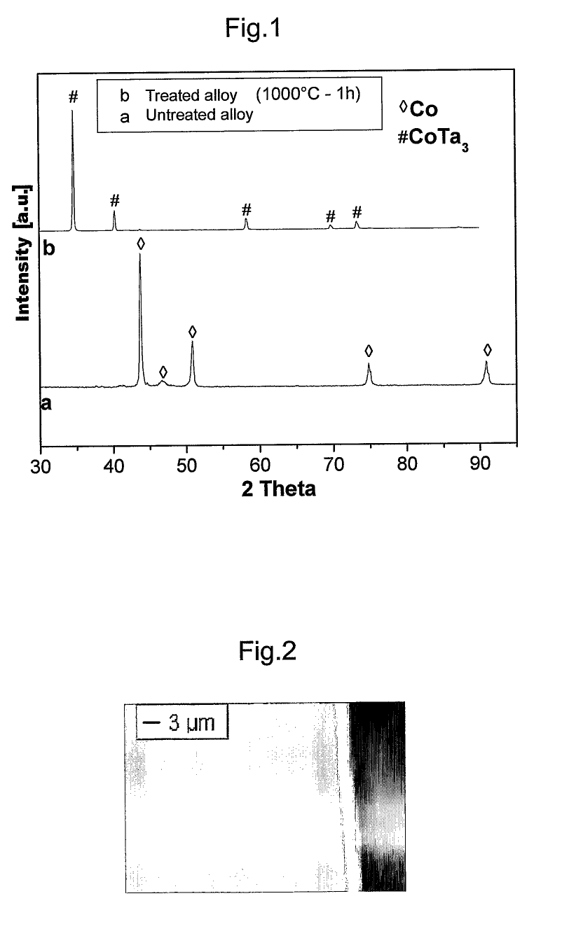

FIG. 1 is a x-ray diffraction spectrum, relative to the untreated substrate of a cobalt alloy, employed in the example, and relative to the same alloy after the treatment reported in the invention; and

FIG. 2 is a SEM-BS image of the section of the sample according to the example.

### EXAMPLE

The BIODUR CCM PLUS alloy (Carpenter Technology Corporation) was used as substrate. The surface modification treatment consists of a thermal treatment in molten salts at 1000° C. for one hour, without applying any electrical field (the salt mixture includes NaCl 47 wt %, K₂TaF₇ 52 wt % and Ta 1 wt % in powder form).

The substrate of the BIODUR alloy registers a weight increment of 0.02 mg/m², after the surface modification treatment, due to the diffusion of tantalum into the surface.

The untreated alloy shows an austenitic structure. The surface modification involves the formation of a metastable intermetallic compound, which is rich in tantalum (CoTa₃).

The surface composition after the treatment is 81 Co and 19 Ta (at %), which is not far from CoTa₃.

The modified surface layer shows a thickness of about 3 μm, as it can be observed on the SEM picture reported in FIG. 2. The interface with the substrate is continuous, crack and pore free and it follows the surface discontinuity of the substrate.

The roughness value changes from 6 to 40 nm after the treatment and it was measured by using a RANK TAYLOR HOBSON instrument for the surface profile analysis with a laser inter-pherometric unit. So the roughness of the treated material is low and it is acceptable according to the international standards for the metallic surfaces of joints (ISO-7206-2).

The treated surface shows a better wettability respect to the untreated one; the contact angles go down from 80° to 46° after the treatment.

The friction coefficient is lower in the case of the treated alloy respect to the untreated one both in the case of a contact of treated material on treated material (pin-on-disc test), and in the case of treated material on alumina (pin-on-ball test).

It is about 0.32 in the first case (pin-on-disc), when considering untreated material, and about 0.24 when considering the treated one. In the second case (ball-on-disc) it goes down from 0.23 to 0.18 after the treatment.

The abrasive wear is 0.755·10⁻⁴ [mm³/Nm], measured by a ball on disc test performed by using an alumina ball and a disc of cobalt alloy, at a contact pressure of 1.7 GPa, in the case of treated material, while it is 5.723·10⁻⁴ [mm³/Nm] before the treatment.

The depth of the wear track after the ball-on-disc test is lower respect to the thickness of the surface modified layer and the presence of tantalum was still revealed on the disc inside the track.

This test is drastically more restrictive respect to the conditions during natural walking (maximum stress of about 5 MPa). So it can be concluded that the modified BIODUR alloy satisfies the requirements as a wear resistant barrier presenting high biocompatibility.

Furthermore the material shows an increment of hardness (from 493 HV up to 557 HV) due to the surface modification treatment.

The invention concerns medical prosthetic devices including all the devices for implantation in human or animal body made out of cobalt or a cobalt alloy, as before suggested, as particularly articular prostheses with a metal-on-metal or a metal-on-polyethylene contact, as for instance total hip or knee joints, femoral heads, acetabular cups and articular inserts. So its main use is in devices submitted to wear and it is not for direct bone contact.

### BIBLIOGRAPHY

[1] G. Gasparini, G. Meccauro, E. Espa, T. Nizegorodcew in Risposta Biologica alla Formazione di Detriti Metallici nelle Artroprotesi di Anca non Cementate, XII Congresso della Societa Italiana di Ortopedia e Traumatologia (SIBOT) (1999);
[2] P. Rossi, P. Sibelli, F. Castoldi, P. Rossi in Miti e Realtà nell'Accoppiamento dei Biomateriali nell'Anca, XIV Congresso della Societa Italiana di Ortopedia e Traumatologia (SIBOT) (2001);
[3] A. Massè, M. Bosetti, C. Buratti, O. Visentin, D. Bergadano, M. Carras in J. Biom. Mat. Res., B, 67, 750 (2003);
[4] D. M. Findlay, K. Welldon, G. J. Atkins et al. in Biomat., 25, 2215 (2004);
[5] X. Zou, H. Li, M. Bünger, N. Egund, M. Lind, C. Bünger in The Spine Journal, 4, 99 (2004);
[6] D. M. Findlay, K. Welldon, G. J. Atkins, D. W. Howie, A. C. W. Zannettino, D. Bobyn in Biomaterials, 25, 2215 (2004);
[7] H. Matsuno, A. Yokoyama, F. Watari, M. Uo, T. Kawasaki in Biomaterials, 22, 1253 (2001).

1. Medical prosthetic device, including a bulk made out of cobalt or a cobalt alloy, characterised by a thin surface layer enriched in tantalum and/or niobium, which includes Co—Ta and/or Co—Nb intermetallic compounds

2. Prosthetic device according to claim 1, characterised in that said surface layer has a thickness of 0.5-40 μm.

3. Prosthetic device according to claim 1, characterised in that said surface layer shows a surface content of tantalum and/or niobium of 0.5-90 wt %.

4. Prosthetic device according to claim 3, characterised in that said surface layer shows a concentration of tantalum and/or niobium of 70-90 wt %.

5. Prosthetic device according to claim 1 characterised in that the bulk is made out of a cobalt alloy containing a cobalt amount higher than 40 wt %.

6. Prosthetic device according to claim 5, characterised in that this bulk is made out of a cobalt alloy containing 45-70 wt % of cobalt and 15-40 wt % of chromium, and the balance to 100 is one or more elements selected from the group consisting of tungsten, molybdenum, nickel, iron, silicon, manganese and carbon.

7. Prosthetic device according to claim 1, characterised in that said surface layer is enriched in tantalum and/or niobium and it can be obtained through a treatment of the bulk of the prosthetic device in a mixture of molten salts, without applying any electrical current, containing a tantalum or niobium halide, respectively, and optionally tantalum and or niobium in powder form, at a temperature of 700° C.-1500° C.

8. Prosthetic device according to claim 1, characterised in that said surface layer shows an enhanced hardness respect to the hardness of the cobalt or cobalt alloy of the bulk.

9. Prosthetic device according to the claim 8, characterised in that said surface layer shows an increment of hardness from 10% up to 40% respect to the hardness of the material of the bulk.

10. Prosthetic device according to claim 1, characterised in that said surface layer shows an abrasive wear significantly lower than the abrasive wear of the cobalt or cobalt alloy of the bulk.

11. Prosthetic device according to claim 1, constituted by an articular prosthesis, in particular the femoral head and/or the acetabular cup of a hip or knee joint presenting a metal-on-metal or a metal-on-polyethylene (UHMWPE) contact.

12. Process for the production of a medical prosthetic device with enhanced biocompatibility, hardness and wear resistance, including a bulk made out of cobalt or a cobalt alloy, characterised in that it includes a treatment of said bulk in a mixture of molten salts, containing a tantalum and/or niobium halide, eventually in the presence of metallic tantalum and/or niobium at a temperature of 700° C.-1500° C., without applying any electrical field, in order of inducing the formation of a thin surface layer enriched in tantalum and/or niobium, constituted by intermetallic compounds.

13. Process according to the claim 12, characterised in that
said molten salt mixture includes 20-100 wt % of a tantalum and/or niobium halide and 0-80 wt % of an alkaline or alkaline earth halide.

14. Process according to claim 13, characterised in that said molten salt mixture includes tantalum and/or niobium up to 20 wt % in powder form respect to the composition of the molten salt mixture.

15. Process according to claim 12, characterised in that said treatment is performed at a temperature higher than 800° C. and during a time of 15 minutes up to 8 hours without applying any electrical field.

16. Process according to claim 12, characterised in that said bulk of the prosthetic device is made out of a cobalt alloy containing at least 40 wt % of cobalt.
